(19) United States
(12) Patent Application Publication
(10) Pub. No.: US 2008/0081036 A1
(43) Pub. Date: Apr. 3, 2008

(54) FORMULATION AND METHOD FOR A SHAMPOO FOR ANIMALS

(75) Inventors: Thomas W. Dann, Oldsmar, FL (US); Michael C. Fleck, Bradenton, FL (US)

Correspondence Address: DLA PIPER US LLP, 4365 EXECUTIVE DRIVE, SUITE 1100, SAN DIEGO, CA, 92121-2133, US

(73) Assignee: Epiderma Pet, LLC, Bradenton, FL (US)

(21) Appl. No.: 11/852,076

(22) Filed: Sep. 7, 2007

Related U.S. Application Data
(60) Provisional application No. 60/843,375, filed on Sep. 8, 2006.

(51) Int. Cl. A61K 38/48; A61K 31/045; A61K 31/17; A61K 31/195; A61K 31/215; A61K 31/355; A61K 31/4164; A61P 17/00; A61K 35/64; A61K 36/00; A61K 36/02; A61K 36/42; A61K 36/534; A61K 36/82

(52) U.S. Cl. 424/94.65; 424/195.17; 424/537; 424/725; 424/729; 424/747; 424/758; 424/775; 424/778; 514/23; 514/400; 514/458; 514/546; 514/558; 514/561; 514/588; 514/738; 514/789

(57) ABSTRACT

Animal shampoo compositions and methods of using the same for topical treatment of pets in need of improvement of quality of skin are provided. Methods for preparing such shampoos are also provided.

### CROSS-REFERENCE TO RELATED APPLICATIONS

This application claims priority under 35 U.S.C. §119(e) to U.S. Patent Application Ser. No. 60/843,375 filed Sep. 8, 2006, which is herein incorporated by reference in its entirety.

### FIELD OF THE INVENTION

The present invention relates generally to animal skin care products and more specifically to animal shampoos and methods of using the same for topical treatment of pets in need of improvement of quality of skin.

### BACKGROUND

Many domestic animals, including dogs and cats, frequently suffer from dry, oily, itchy, or flaky skin, or from similar skin defects, maladies, or imperfections. The skin of the animals is also often malodorous. Previously, many products, such as shampoos, fragrances and the like have been developed for treating at least some of such conditions. Mostly, such products were of a cosmetic nature and failed to effectively treat the underlying sources of such maladies. At best, only limited and temporary relief was achieved using such traditional products.

The nature of animal skin differs substantially from that of human skin in many respects. More specifically, human skin is quite acidic having a pH of about 5.2, while the animal skin is essentially neutral and has a pH of about 7.0. In addition, animal skin is much thicker than human skin and is covered with a layer of hair that is also much thicker. Accordingly, the methods of treatment of human skin having the above-described problems cannot be easily adopted for the treatment of animal skin.

It is very desirable to have a product that can remedy all or many of the above-described problems and to reduce dandruff, neutralize and/or eliminate oil from the skin, soften and brighten the hair coat, moisturize the skin, repel insects, de-odorize the skin and add a pleasant fragrance to it, as well as to serve as a high quality animal shampoo. Currently, such a product has not been provided. The present application provides the formulations that achieve these goals and describes the methods of using such compositions.

### SUMMARY

The present invention provides shampoo compositions for treating an animal having a skin in need of improvement of the condition thereof, the composition comprising at least one humectant or moisturizer, at least one surfactant, at least one skin conditioner, at least one hair conditioner, at least one cleansing agent, at least one exfoliant, at least one oil, at least one antioxidant, at least one preservative, at least one emollient, at least one fragrance; and at least one astringent.

The present invention further provides methods for preparing such shampoo compositions as well as methods for using the compositions for improving the skin of a domestic animal such as a dog, a cat, or a horse.

### BRIEF DESCRIPTION OF DRAWINGS

FIGS. 1-3 demonstrate schematically a method of treating a domestic animal according to an embodiment of the present invention.

### DETAILED DESCRIPTION

The following are the definitions of some terms as used in the present application. If the terms and definitions provided below are inconsistent in any way with their generally accepted meanings, then, only to the extent of such potential inconsistencies, the meanings provided below prevail.

The term “a humectant” is considered synonymous with the term “a moisturizer” and refers to a substance designed to make softer and more pliable the epidermis (i.e., the external layers of the skin), by increasing the extent of its hydration, allowing the addition of water to the epidermis, or the retention of water by the epidermis, or both.

The term “a surfactant” refers to a surface-active substance, i.e., a substance capable of reducing the surface tension of a liquid in which it is dissolved.

The term “an astringent” refers to an agent that is capable of causing tissues contract or shrink.

The term “an exfoliant” refers to an agent that helps to remove dead skin cells on the skin's surface, thereby improving skin tone.

The term “an emollient” refers to an agent that is capable of softening and soothing the skin when applied locally.

The terms “a skin conditioner” and “a hair conditioner” refer to a substance that improves the quality of the skin or hair, respectively, such as improving softness, manageability and luster.

The term “a viscosity adjusting agent” is defined as a substance substantially altering a viscosity of a liquid composition compared to the viscosity of the composition in the absence of such substance.

The term “topically” refers to direct application of a substance on the skin or hair of an animal.

The term “spraying” refers to a process of the application of a liquid in the form of small particles ejected from a spraying device.

The term “aerosol” refers to a suspension of liquid particles in air.

According to an embodiment of the present invention, a shampoo composition is provided. The composition can include at least one humectant or moisturizer, at least one surfactant, at least one skin conditioner, at least one hair conditioner, at least one cleansing agent, at least one exfoliant, at least one oil, at least one antioxidant, at least one preservative, at least one emollient (soothing agent), at least one astringent, a fragrance(s), and water.

Some humectants that can be used can also serve as a hair conditioner, and/or a skin conditioner. Some surfactants that can be used can also serve as a hair conditioner, and/or as a foam booster, and/or as a cleansing agent. Some hair conditioners that can be used can also serve as a skin conditioner. Some oils that can be used can also serve as a skin conditioner. Some emollients that can be used can also serve as a skin conditioner. Some antioxidants that can be used can also serve as a skin conditioner. Some astringents that can be used can also serve as a skin conditioner.

Optionally, the composition can also be formulated using a viscosity adjusting agent, e.g., sodium chloride. Optionally, the composition can also be formulated using any commonly used buffer system, if maintaining a certain level of pH is necessary. For example, citric acid can be used to adjust pH.

The total concentration of the moisturizer(s) in the composition can be between about 1 and 10 mass % of the total composition. Some non-limiting examples of moisturizers that can be used include glycerin, honey, and algae extracts. Other non-limiting examples of moisturizers that can be used include urea, sodium lactate, and some amino acids, such as glycine or histidine.

The total concentration of the cleansing agent(s) in the composition can be between about 25 and 40 mass % of the total composition. Some non-limiting examples of cleansing agents that can be used include sodium laurate sulfate, and PEG-80 sorbitan laurate.

The total concentration of the surfactant(s) in the composition can be between about 10 and 20 mass % of the total composition. Some non-limiting examples of surfactants that can be used include sodium C₁₄₋₁₆ olefin sulfonate, disodium cocamphodiacetate, and PEG-80 sorbitan laurate.

The total concentration of the skin conditioner(s) in the composition can be between about 2 and 15 mass % of the total composition. Some non-limiting examples of skin conditioners that can be used include glycerin, wheat amino acid, lavandula angustifolia (lavender) extract, PEG-120 methyl glucose trioleate, honey, mentha pulegium extract, cucumis sativus (cucumber) fruit extract, camellia simensis leaf extract, chamomilla recutita (matricaria) flower extract, rosamarinus officinalis (rosemary) leaf extract, tocopheryl acetate, algae extract, and hamamelis virginiana (witch hazel).

The total concentration of the hair conditioner(s) in the composition can be between about 2 and 10 mass % of the total composition. Some non-limiting examples of hair conditioners that can be used include glycerin, disodium cocamphodiacetate, and wheat amino acid.

The total concentration of the exfoliant(s) in the composition can be between about 0.1 and 1 mass % of the total composition. One non-limiting example of an exfoliant that can be used is bromelain.

The total concentration of the oil(s) in the composition can be between about 0.1 and 2 mass % of the total composition. Some non-limiting examples of an oil that can be used include lavandula angustifolia (lavender) extract and cedrus atlantica (cedarwood) bark oil.

The total concentration of the antioxidant(s) in the composition can be between about 0.1 and 3 mass % of the total composition. Some non-limiting examples of antioxidants that can be used include melaleuca altermifolia (tea tree) leaf oil, camellia simensis leaf extract, and tocopheryl acetate.

The total concentration of the preservative(s) in the composition can be between about 0.1 and 1 mass % of the total composition. Some non-limiting examples of preservatives that can be used include methylisothiazolinone, and methylchloroisothiazolinone.

The total concentration of the emollient(s) in the composition can be between about 0.1 and 2 mass % of the total composition. Some non-limiting examples of emollients that can be used include PEG-120 methyl glucose trioleate, and cucumis sativus (cucumber) fruit extract.

The total concentration of the astrigent(s) in the composition can be between about 0.1 and 1 mass % of the total composition. One non-limiting example of an astrigent that can be used is hamamelis virginiana (witch hazel).

Any composition satisfying the above-described requirements can be prepared using common formulating techniques known to those having ordinary skill in the art. For example, the above-described components can be mixed with one another, followed by adding water, to form an aqueous composition, e.g., by employing rapid stirring. Alternatively, each component, in a separate container, can be preliminary dissolved in water, or otherwise mixed with water resulting in a plurality of water-based systems, each contained in a separate container. The contents of all the containers can then be combined, e.g., by stirring or shaking, to form the final composition.

If desired, those having ordinary skill in the art can design other methods of mixing the components forming the composition. Regardless of the method of mixing that is selected, those having ordinary skill in the art will provide such quantities of each component so that the concentration of each of the components in the composition satisfies the above-described limits.

A method for the treatment of an animal is further provided. A composition can be prepared according to a procedure described above, followed by washing the animal in a common way. The composition can be then applied topically onto the skin of an animal that needs the improvement of its skin condition. Various methods can be used for applying the composition onto the skin of an animal. For example, the composition can be sprayed using a conventional hand-operated pump. Alternatively, the composition can be formulated to form an aerosol using commonly known methods for aerosol preparation. Those having ordinary skill in the art can devise other methods for applying the composition.

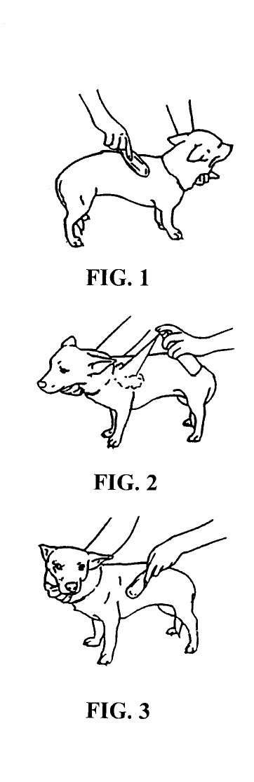

Referring now to FIGS. 1-3, one exemplary procedure that can be utilized for the treatment of a dog can be briefly described as follows. A suitable brush can be used for the procedure below, such as a two-sided brush that has metal bristles on one side and nylon-type bristles on the other. Using such or similar brush, the hair coat and skin of a dog can be energetically brushed using the metal side of the brush (FIG. 1). The duration of brushing can be at least 30 seconds.

Following the brushing procedure, the composition can be sprayed onto the entire body of the dog (FIG. 2). A hand-pump can be used for spraying. Alternatively, the composition can be applied using an aerosol generator, such as an aerosol can. At least 10 sprays can be used. The heaviest spraying can be applied over the back and sides of the dog. Fewer sprays can be applied for a smaller dog, and more sprays can be used if the dog is larger. The product should not be sprayed into the dog's eyes. Instead, the product can be applied to the brush first, followed by gentle brushing over the head and face.

When the product has been sprayed on, the product can then be brushed into the hair coat and skin of the dog using the metal side of the brush (FIG. 3). The duration of brushing can be at least 30 seconds. Then, the brush head can be reversed and the hair coat can be gently smoothed over. The soft side of the brush can be used for brushing the head and underside of the dog. If the dog's skin is red and sensitive, the softer side of the brush can be used. The entire procedure can be repeated daily.

A variety of animals can be treated according to the methods of the present invention. In addition to dogs, as disclosed in the above-described exemplary procedure, some non-limiting examples of other animals that can be similarly treated can also include other domestic pets such as cats, as well as horses, cows, sheep, goats, guinea pigs, rats or other bovine, ovine, equine, canine, feline, or rodent species. Those skilled in the art can adjust the treatment procedure taking in account the nature of the animal to be treated.

The following examples are provided to further illustrate the advantages and features of the present invention, but are not intended to limit the scope of the invention.

### EXAMPLE 1

### Preparation and Use of Composition # 1

Composition # 1 can be prepared by mixing, by energetic stirring for about three hours, the following components in the quantities shown below:

(a) between about 25 and 30 mass % of sodium lauryl sulfate;

(b) between about 10 and 15 mass % of sodium C₁₄₋₁₆ olefin sulfonate;

(c) between about 2 and 5 mass % of glycerin;

(d) between about 1 and 3 mass % of disodium cocamphodiacetate;

(e) between about 1 and 3 mass % of PEG-80 sorbitan laurate;

(f) between about 0.1 and 1 mass % of wheat amino acid;

(g) between about 0.1 and 1 mass % of a fragrance;

(h) between about 0.1 and 1 mass % of bromelain;

(i) between about 0.1 and 1 mass % of lavandula angustifolia (lavender) extract;

(j) between about 0.1 and 1 mass % of melaleuca altermifolia (tea tree) leaf oil;

(k) between about 0.1 and 0.5 mass % of methylisothiazolinone;

(l) between about 0.1 and 0.5 mass % of methylchloroisothiazolinone;

(m) between about 0.1 and 2 mass % of honey;

(n) between about 0.1 and 1 mass % of mentha pulegium extract;

(o) between about 0.1 and 1 mass % of cucumis sativus (cucumber) fruit extract;

(p) between about 0.1 and 1 mass % of cedrus atlantica (cedarwood) bark oil;

(q) between about 0.1 and 1 mass % of camellia simensis leaf extract;

(r) between about 0.1 and 1 mass % of chamomilla recutita (matricaria) flower extract;

(s) between about 0.1 and 1 mass % of algae extract;

(t) between about 0.1 and 1 mass % of hamamelis virginiana (witch hazel);

(u) optionally, between about 0.1 and 2 mass % of PEG-120 methyl glucose trioleate;

(v) optionally, between about 0.1 and 2.5 mass % of citric acid;

(w) optionally, between about 0.1 and 2 mass % of sodium chloride;

(x) optionally, between about 0.1 and 1 mass % of rosamarinus officinalis (rosemary) leaf extract;

(y) optionally, between about 0.1 and 1 mass % of tocopheryl acetate; and

(z) the balance, water.

The resulting composition # 1 can be applied on a domestic animal in need of improvement of its skin condition, such as a dog or a cat, using the procedure described above and illustrated by FIGS. 1-3, following by washing of the animal.

Although the invention has been described with reference to the above example, it will be understood that modifications and variations are encompassed within the spirit and scope of the invention. Accordingly, the invention is limited only by the following claims.

What is claimed is:

1. A method for treating an animal having a skin in need of improvement of the condition thereof, the method comprising topically administering to the animal an effective amount of a shampoo composition comprising:
(a) at least one humectant or moisturizer;
(b) at least one surfactant;
(c) at least one skin conditioner;
(d) at least one hair conditioner;
(e) at least one cleansing agent;
(f) at least one exfoliant;
(g) at least one oil;
(h) at least one antioxidant;
(i) at least one preservative;
(k) at least one emollient;
(k) at least one fragrance;
(l) at least one astringent; and
(m) a solvent,
thereby treating the animal, wherein the animal is selected from a group consisting of a dog, a cat, and a horse.

2. The method of claim 1, wherein the topical administration comprises:
(a) applying the composition onto the skin of the animal by spraying the composition using a pump, or
(b) using an aerosol generator, creating an aerosol from the composition, followed by applying the aerosol onto the skin of the animal and brushing the skin of the animal.

3. The method of claim 1, wherein the humectant or moisturizer is selected from the group consisting of glycerin, honey, an algae extract, urea, sodium lactate, glycine, and histidine.

4. The method of claim 1, wherein the surfactant is selected from the group consisting of sodium C₁₄₋₁₆ olefin sulfonate, disodium cocamphodiacetate, and PEG-80 sorbitan laurate.

5. The method of claim 1, wherein the skin conditioner is selected from the group consisting of glycerin, wheat amino acid, lavandula angustifolia (lavender) extract, PEG-120 methyl glucose trioleate, honey, mentha pulegium extract, cucumis sativus (cucumber) fruit extract, camellia simensis leaf extract, chamomilla recutita (matricaria) flower extract, rosamarinus officinalis (rosemary) leaf extract, tocopheryl acetate, algae extract, and hamamelis virginiana (witch hazel).

6. The method of claim 1, wherein the hair conditioner is selected from the group consisting of glycerin, disodium cocamphodiacetate, and wheat amino acid.

7. The method of claim 1, wherein the cleansing agent is selected from the group consisting of sodium laurate sulfate and PEG-80 sorbitan laurate.

8. The method of claim 1, wherein the exfoliant is bromelain.

9. The method of claim 1, wherein the oil is selected from the group consisting of lavandula angustifolia (lavender) extract and cedrus atlantica (cedarwood) bark oil.

10. The method of claim 1, wherein the antioxidant is selected from the group consisting of melaleuca altermifolia (tea tree) leaf oil, camellia simensis leaf extract, and tocopheryl acetate.

11. The method of claim 1, wherein the preservative is selected from the group consisting of methylisothiazolinone and methylchloroisothiazolinone.

12. The method of claim 1, wherein the emollient is selected from the group consisting of PEG-120 methyl glucose trioleate and cucumis sativus (cucumber) fruit extract.

13. The method of claim 1, wherein the astringent is hamamelis virginiana (witch hazel).

14. A shampoo composition for treating an animal having a skin in need of improvement of the condition thereof, the composition comprising:
(a) at least one humectant or moisturizer;
(b) at least one surfactant;
(c) at least one skin conditioner;
(d) at least one hair conditioner;
(e) at least one cleansing agent;
(f) at least one exfoliant;
(g) at least one oil;
(h) at least one antioxidant;
(i) at least one preservative;
(j) at least one emollient;
(k) at least one fragrance;
(l) at least one astringent; and
(m) a solvent.

15. The composition of claim 14, wherein the humectant or moisturizer is selected from the group consisting of glycerin, honey, an algae extract, urea, sodium lactate, glycine, and histidine.

16. The composition of claim 14, wherein the surfactant is selected from the group consisting of sodium C₁₄₋₁₆ olefin sulfonate, disodium cocamphodiacetate, and PEG-80 sorbitan laurate.

17. The composition of claim 14, wherein the skin conditioner is selected from the group consisting of glycerin, wheat amino acid, lavandula angustifolia (lavender) extract, PEG-120 methyl glucose trioleate, honey, mentha pulegium extract, cucumis sativus (cucumber) fruit extract, camellia simensis leaf extract, chamomilla recutita (matricaria) flower extract, rosamarinus officinalis (rosemary) leaf extract, tocopheryl acetate, algae extract, and hamamelis virginiana (witch hazel).

18. The composition of claim 14, wherein the hair conditioner is selected from the group consisting of glycerin, disodium cocamphodiacetate, and wheat amino acid.

19. The composition of claim 14, wherein the cleansing agent is selected from the group consisting of sodium laurate sulfate and PEG-80 sorbitan laurate.

20. The composition of claim 14, wherein the exfoliant is bromelain.

21. The composition of claim 14, wherein the oil is selected from the group consisting of lavandula angustifolia (lavender) extract and cedrus atlantica (cedarwood) bark oil.

22. The composition of claim 14, wherein the antioxidant is selected from the group consisting of melaleuca altermifolia (tea tree) leaf oil, camellia simensis leaf extract, and tocopheryl acetate.

23. The composition of claim 14, wherein the preservative is selected from the group consisting of methylisothiazolinone and methylchloroisothiazolinone.

24. The composition of claim 14, wherein the emollient is selected from the group consisting of PEG-120 methyl glucose trioleate and cucumis sativus (cucumber) fruit extract.

25. The composition of claim 14, wherein the astringent is hamamelis virginiana (witch hazel).
